(19) United States
(12) Patent Application Publication
(10) Pub. No.: US 2011/0201111 A1
(43) Pub. Date: Aug. 18, 2011

(54) METHOD FOR OBTAINING CONNECTIVE MESENCHYMAL STEM CELLS FROM THE MONONUCLEAR FRACTION OF HUMAN BONE MARROW

(75) Inventors: Joan Garcia Lopez, Barcelona (ES); Luciano Rodriguez Gomez, Barcelona (ES); Jordi Joan Cairo Badillo, Barcelona (ES); Arnau Pla Calvet, Barcelona (ES); Francesc Godia Casablancas, Barcelona (ES)

(73) Assignee: BANC DE SANG I TEIXITS, Barcelona (ES)

(21) Appl. No.: 13/124,916

(22) PCT Filed: Nov. 25, 2009
(86) PCT No.: PCT/ES2009/000547

§ 371 Date: Apr. 19, 2011

(30) Foreign Application Priority Data
Jan. 2, 2009 (ES) P200900005

(51) Int. Cl. C12N 5/0775

(52) U.S. Cl. 435/372

(57) ABSTRACT

The present invention relates to a method for obtaining connective mesenchymal stem cells from the mononuclear fraction of human bone marrow cells using type AB human serum as a supplement. Said method includes the steps of obtaining said mononuclear fraction of bone marrow, the step of recovering and pre-expanding the connective stem cells and the step of expanding the connective stem cells until obtaining the clinical dose required for the therapeutic use thereof.

This invention relates to a method for obtaining mesenchymal stem cells. More specifically, this invention relates to a method for obtaining mesenchymal stem cells from the mononuclear fraction of human bone marrow cells. The said method comprises the stages of obtaining the said mononuclear bone marrow fraction, the stage of recovery and pre-expansion of the mesenchymal stem cells, and the stage of expanding the mesenchymal stem cells until the clinical dose necessary for possible therapeutic use is obtained.

Mesenchymal stem cells (MSC) are multipotent cells that have demonstrated their effectiveness in the treatment of musculoskeletal lesions and in improving heart function in cardiovascular diseases (Le Blanc K., Pittenger M., Mesenchymal stem cells: progress toward promise. Cytotherapy 2005; 7: 36-45). MSC may be isolated from various adult tissues, including bone marrow, adipose tissue and umbilical cord blood. In addition to this, these cells can be expanded ex vivo.

The mononuclear bone marrow fraction (MMF) is obtained by means of a separating operation based on the centrifugation of blood obtained from bone marrow. MMF is a heterogeneous mass of cells which in addition to mesenchymal stem cells may include hematopoietic progenitor cells. These two populations are found in a very small concentration in relation to mature hematopoietic cells, which are also present in MMF.

Fridenshtein et al. (Fridenshtein A. J., Deriglazova U. F., Kulagina N. N. et al. Precursors for fibroblasts in different populations of hematopoietic cells as detected by the in vitro colony assay method. Exp. Hematol. 1974 Vol. 2. P. 83-92) obtained mesenchymal stem cells from bone marrow for the first time, basing their technique on the ability of mesenchymal stem cells to adhere to plastics surfaces, a property which most hematopoietic cells do not have. The disadvantage of this method is the low yield obtained when recovering mesenchymal cells, which can be attributed to the low prevalence of the cell type in bone marrow, estimated at between 0.001 and 0.01% (Alhadlag A. and Mao J. Mesenchymal Stem Cells: Isolation and Therapeutics. Stem Cells and Development. 2004. 13: 436-448 (3). Nevertheless adhesion to plastics has been established as the starting point for further improvement in methods for obtaining mesenchymal stem cells.

Heynesworth et al. have succeeded in obtaining mesenchymal stem cells with high capacity for adhesion, a high proliferation level and maintained multipotency over a long period of time (Heynesworth S. E., Goshima J., Goldberg V. M., Calplan A. I. Characterization of cells with osteogenic potential from the human bone marrow//Bone. 1995. Vol. 13. P. 81-85) using bovine foetal serum as a supplement for the culture medium. The disadvantage of this method lies in the fact that searching for a suitable lot for cell culture and corresponding analysis of the bovine foetal serum takes a great deal of time and is very onerous. Furthermore, because of the great variation from lot to lot shown by bovine foetal serum, it is not possible to guarantee that results will be reproducible.

Another disadvantage of this method is that in order to obtain sufficient quantities of mesenchymal stem cells for clinical use the cells must be cultured for between 4 and 6 weeks, and in some circumstances, such as in patients with diseases of bone metabolism, the culture time may be longer. The reason why such long culture times are necessary is, as mentioned previously, the small quantity of mesenchymal cells which it is possible to obtain from bone marrow.

A long cell culture time can increase contamination risk and considerably increase the cost of maintaining a culture. Furthermore as the number of duplications increases there is a risk that the cells might lose multipotency or enter into senescence, which would compromise their therapeutic capacity.

The approach normally used to shorten mesenchymal cell culture times centres attention on factors such as supplements for the culture medium, the nature of the plastics surface, the inoculating dose and the culture means used (Sotiropolou P. A., Pérez S. A., Salagianni M, Baxevanis C. N., Papamichail M. Characterization of the optimal culture conditions for clinical scale production of human mesenchymal stem cells. Stem Cells 2006; 24: 462-471).

One object of this invention is to provide a method for the ex vivo culture of mesenchymal stem cells from human bone marrow in which the time and number of duplications necessary to obtain a clinical dose of mesenchymal stem cells are reduced in comparison with conventional methods. This procedure is based on the synergistic effect of combining very specific conditions for the isolation of mesenchymal cells from mononuclear bone marrow fraction with the use of human serum of blood group AB as a supplement for the culture medium. This is used as a replacement for the costly and poorly-reproducible bovine foetal serum. Furthermore human AB serum has the advantages that it is more economical and offers better reproducibility than bovine foetal serum. In turn the proposed isolation conditions make it easy to recover a larger number of mesenchymal cells from the mononuclear fraction in comparison with the conditions used in conventional strategies. This fact is particularly relevant when it is borne in mind that the higher the initial number of available isolated mesenchymal cells the shorter the time and resources required to obtain the required clinical dose.

These isolation conditions have been achieved through a systematic study of their effect on the recovery of mesenchymal cells from inoculum and the washing time for mononuclear bone marrow cells in culture media supplemented with human AB serum.

Another object of this invention is to provide a method which makes it possible to have an identical back-up copy of the mesenchymal stem cells, with the same characteristics as the initial mesenchymal stem cells as regards ability to proliferate and differentiate.

The method according to this invention can be applied to the acquisition of mesenchymal cells from other sources such as adipose tissue, the pancreas, liver, skeletal muscle, dermis, synovial membrane, trabecular bone, umbilical cord blood, pulmonary tissue, dental pulp and periodontal connective tissue. In the case where the source of mesenchymal cells is not bone marrow the initial treatment will differ appropriately for each cell type used as a source.

### DETAILED DESCRIPTION OF THE INVENTION

As a consequence, one object of this invention is to provide a method for obtaining mesenchymal stem cells from the mononuclear fraction of human bone marrow, obtained by centrifugation with a density gradient, characterised in that it comprises the stages of:

a) Recovery and pre-expansion of the mesenchymal stem cells through a primary culture in which the mononuclear bone marrow cells are inoculated into DMEM culture medium supplemented with human AB serum (10%) following the culture protocol below:
the mononuclear culture cells are washed with saline solution on day 5 and culture medium supplemented with human AB serum (10%) is added,
the culture medium is replaced after 9 days by the same medium as mentioned above,
the primary culture is trypsinised at 12 days. These cells generate the expansion culture (c),

b) Secondary culture: reinoculation of the mononuclear cells washed in (a) in culture medium supplemented with human AB serum (10%) following the culture protocol below:
the culture is washed with saline solution on day 5 and culture medium supplemented with human AB serum (10%) is added,
the culture medium is replaced by the same culture medium as mentioned above on day 9,
the primary culture medium is trypsinised on day 12. The mesenchymal stem cells, which are frozen and stored in nitrogen containers, are recovered,

c) Expansion culture: expansion of the mesenchymal cells obtained in a) on day 12. These cells are reinoculated and expanded in culture medium supplemented with human AB serum (10%) following the culture protocol below:
the culture medium is replaced by the same medium as mentioned above on days 3 and 6,
the culture medium is trypsinised on day 12. These cells are packed for transfer to the operating theatre.

In a preferred embodiment between 200,000 and 400,000 cells per square centimetre of surface area are inoculated in the primary culture. Furthermore the culture medium used for both the primary culture and the secondary culture (stage b) is preferably Dulbecco modified Eagle (DMEM) with a supplement of 10% (v/v) human AB serum.

The method according to this invention is advantageous in that a high number of mesenchymal stem cells, between 40.10⁶ and 50.10⁶, are obtained with a low number of duplications, between 6 and 8, and in a shorter period of time (around 20 days) than in conventional processes.

Furthermore an additional advantage is that by freezing the mesenchymal stem cells obtained in the secondary culture (stage b)) it is possible to generate the required dose of cells should the method fail for accidental reasons, or to have an identical back-up copy of the mesenchymal stem cells.

The method according to this invention also has the additional advantages of simple manipulation and low costs, in that neither extensive culture nor the use of bovine foetal serum are required.

This invention is described below in greater detail with reference to an example. This example is not however intended to restrict the technical scope of this invention.

### Example 1

300,000 mononuclear bone marrow cells per square centimetre obtained by the gradient centrifugation of an extract of bone marrow blood were inoculated into a commercial culture flask of 636 cm² containing DMEM medium supplemented with human AB serum (10%). The culture was washed after 5 days and the supernatant was inoculated into a second commercial culture flask of 636 cm² which contained DMEM medium supplemented with human AB serum (10%). Culture medium (DMEM supplemented with human AB serum (10%)) was added to the culture medium, with a culture area to medium volume ratio of 3:1. On day 9 the culture medium was replaced and culture medium (DMEM supplemented with human AB serum (10%)) was added with a culture area/medium volume ratio of 3:1. On day 12 the culture was trypsinised and between 15.10⁶ and 20.10⁶ mesenchymal stem cells were obtained with a purity of around 80%. These cells were inoculated into two commercial culture flasks of 636 cm² containing DMEM medium supplemented with human AB serum (10%), with a density of 4000 cells/cm². The cells were cultured for 8 days, during which two changes of medium were made (days 3 and 6), using DMEM medium supplemented with human AB serum (10%) with a culture area/medium volume ratio of 3:1.

The non-adhering cells in the primary culture which were inoculated into a secondary culture (stage b)) were washed 5 days after the start of secondary culture (stage b)) and the washings were discarded. The culture medium was replaced on day 9 and culture medium (DMEM supplemented with human AB serum (10%)) was added with a culture area/medium volume ratio of 3:1. On day 12 the culture was trypsinised and between 3.10⁶ and 6.10⁶ mesenchymal stem cells were obtained with a purity of around 65%. The cells obtained were frozen in nitrogen tanks.

The cells obtained in the primary culture demonstrated an immunophenotypic profile with the co-expression of markers 105, 90, 73 and a lack of expression of markers CD45, CD31, HLA-DR, normally associated with mesenchymal stem cells originating from bone marrow. Furthermore the cells obtained demonstrated an ability to differentiate from the bone line, a fact which demonstrates that capacity for multipotency had been maintained. On the other hand the cells obtained in the secondary culture (stage b)) showed an immunophenotypic profile with co-expression of markers 105, 90, 73 and a lack of expression of markers CD45, CD31, HLA-DR, normally associated with mesenchymal stem cells originating from bone marrow. In addition to this the cells obtained showed an ability to differentiate from the bone and adipose lineage, a fact which demonstrates capacity for multipotency had been maintained.

Although the invention has been described with respect to examples of preferred embodiments, these are not to be regarded as restricting the invention, which will be defined by the broadest interpretation of the following claims.

1. A method for culturing mesenchymal stem cells from a mononuclear fraction of human bone marrow comprising:
a) Recovery and pre-expansion of mesenchymal stem cells in a primary culture of DMEM supplemented with human AB serum (10%):
i. washing the mononuclear cell culture of step a) with saline solution on or after 5 days and inoculating the supernatant in DMEM culture medium supplemented with human AB serum (10%),
ii. replacing the culture medium on or after 9 days with DMEM culture supplemented with human AB serum (10%),
iii. the primary culture is trypsinised on day 12,
b) Isolating non-adhering cells from the primary culture of a) and re-inoculating them in DMEM culture medium supplemented with human AB serum (10%):
i. the culture is washed with saline solution on or after day 5, and the supernatant is inoculated in DMEM culture medium supplemented with human AB serum (10%),
ii. the culture medium is replaced on or after 9 days with DMEM culture supplemented with human AB serum (10%),
iii. the resulting culture is trypsinised on day 12, and mesenchymal stem cells are recovered and stored,
c) Residual cells from step a) iii. are re-inoculated and expanded in DMEM culture medium supplemented with human AB serum (10%):
i. the same culture medium is replaced on days 3 and 6,
ii. the culture medium is trypsinised on day 12, and mesenchymal stem cells from the medium are recovered and stored.

2. A method according to claim 1, in which the quantity of mononuclear bone marrow cells inoculated in stage (a) is between 200,000 and 400,000 cells per square centimetre of surface area.

3. A method according to claim 1, in which the quantity of mesenchymal stem cells inoculated in stage (b) is between 3000 and 5000 cells per square centimetre of surface area.

4. A method according to claim 1, in which between 15×10⁶ and 20×10⁶ mesenchymal stem cells are obtained in the primary culture from stage (a).

5. A method according to claim 1, in which between 3×10⁶ and 6×10⁶ mesenchymal stem cells are obtained in the secondary culture (stage (b)).

6. A method according to claim 1, in which the purity of the mesenchymal stem cells obtained in the primary culture in stage (a) is about 80%.

7. A method according to claim 1, in which the purity of the mesenchymal stem cells obtained in the secondary culture (stage (b)) is about 65%.

8. A method according to claim 1, in which between 40×10⁶ and 50×10⁶ mesenchymal stem cells are obtained in stage (c).

9. A method according to claim 1, in which the number of cell duplications is between 6 and 8.

10. A method according to claim 1, wherein the total time for obtaining mesenchymal stem cells from the method does not exceed 21 days.

11. A method according to claim 1, in which the secondary culture (stage (b)) is frozen and kept as a back-up copy.
